(19) United States
(12) Patent Application Publication
(10) Pub. No.: US 2025/0261862 A1
(43) Pub. Date: Aug. 21, 2025

(54) Computing Device Having a Button for Multiple Modes of Sensing

(71) Applicant: Google LLC, Mountain View, CA (US)

(72) Inventors: Aditya Vivekanand Nadkarni, Emeryville, CA (US); Pieris Berreitter, San Francisco, CA (US); Hardik Dilipkumar Shah, Fremont, CA (US); Jr-Jay Jhang, New Taipei City (TW)

(21) Appl. No.: 18/857,430

(22) PCT Filed: May 13, 2022
(86) PCT No.: PCT/US2022/029265

§ 371 Date: Oct. 16, 2024

(51) Int. Cl. A61B 5/0205; A61B 5/00; A61B 5/024; A61B 5/0531; A61B 5/28; A61B 5/332; G04G 21/02; G04G 21/08

(52) U.S. Cl. CPC A61B 5/0205; A61B 5/681; G04G 21/025; G04G 21/08; A61B 5/02405; A61B 5/02416; A61B 5/02438; A61B 5/0531; A61B 5/28; A61B 5/332; A61B 2560/0487

(57) ABSTRACT

A computing device can include a housing defining a cavity and a button assembly. The button assembly can include an insert, a first sensor, and a second sensor. The insert is at least partially positioned within a recess defined by the housing. The first sensor is configured to detect actuation of the insert via an input provided by a user. The second sensor is configured to detect whether the user is touching the insert. The computing device can further include one or more processors positioned within the cavity. The one or more processors is configured to obtain, via the first sensor, first data indicative of actuation of the insert, and obtain, via the second sensor, second data indicative of the user touching the insert. The one or more processors can perform a task based, at least in part, on the first data and the second data.

### FIELD

The disclosure relates generally to computing devices having a multipurpose button. More particularly, the disclosure relates to computing devices that are wearable computing devices with a multipurpose button having an electrode that allows for multiple modes of sensing. For example, a wearable computing device can be worn on a wrist of a user and can include a singular button having sensors to detect biometrics of a user and force actuation of the button.

### BACKGROUND

Wearable computing devices (e.g., wrist watches) can include a display screen to display content (e.g., time, date) to a user. Some wearable computing devices can gather data by using sensors, such as optical and capacitive sensors. The data gathered can be associated with activities performed by the user, or regarding the user's biometrics. Such data may include motion data, and/or biometrics data obtained by measuring various biometric characteristics of the user, such as heart rate, perspiration levels, and the like. Additionally, some wearable computing devices can include buttons to capture the user's interaction.

### SUMMARY

Aspects and advantages of embodiments of the disclosure will be set forth in part in the following description, or can be learned from the description, or can be learned through practice of the example embodiments.

In an example embodiment, a computing device is provided. The computing device includes a housing defining a cavity and a button assembly. The button assembly includes an insert (which may function as a button), a first sensor, and a second sensor. The insert is at least partially positioned within a recess defined by the housing. The first sensor is configured to detect actuation of the insert via an input provided by a user. The second sensor is configured to detect whether the user is touching the insert. Additionally, the computing can include one or more processors positioned within the cavity. The one or more processors are configured to obtain, via the first sensor, first data indicative of actuation of the insert. Moreover, the one or more processors are configured to obtain, via the second sensor, second data indicative of the user touching the insert. Furthermore, the one or more processors are configured to perform a task based, at least in part, on the first data and the second data.

In some instances, the insert and the second sensor are one singular component (e.g., the second sensor comprises the insert or consists of the insert, where the insert may function as a button for activating a function of the wearable computing device). For example, the first sensor can be a force sensor and/or the second sensor can be a strain sensor. The second sensor can be an electrode (e.g., strain sensor) that also functions as a button. In an embodiment, the second sensor can be both a strain sensor and an ultrasonic sensor that also functions as a button. In another embodiment, the second sensor can be both a strain sensor and an infrared sensor. In yet another embodiment, the second sensor can be both a strain sensor, an ultrasonic sensor, and/or an infrared sensor.

In an embodiment, the computing device can be a wearable computing device, and the insert (e.g., button and electrode) can be positioned at a periphery of the housing. In an embodiment, the computing device can be a mobile device, and the insert can be positioned at a periphery of the housing of the mobile device or on the upper side of the housing of the mobile device. In an embodiment, the computing device can be a laptop, and the insert can be positioned at a periphery of the housing of the laptop or on a keyboard of the laptop. In an embodiment, the computing device can be a computer tablet, and the insert can be positioned at a periphery of the housing of the computer tablet or on the upper side of the housing of the computer tablet.

In an embodiment, the second sensor is positioned on an input surface of the insert. The second sensor can include a capacitive sensor or be a capacitive sensor. For example, the input surface of the insert forms at least a portion of an electrode of the capacitive sensor. The capacitive sensor can be a dry electrode.

In an embodiment, the insert can be positioned at a periphery of the housing.

In an embodiment, the first sensor or the second sensor can be a piezoelectric sensor, a strain gauge sensor, or an ultrasonic sensor.

In an embodiment, the task performed by the one or more processors can be to measure an electrocardiogram (ECG) reading or an electrodermal activity (EDA) reading.

In some implementations, the task performed can be to reset the computing device. For example, the computing device can be reset when the first data includes an actuation force that is above a force threshold value for at least a specific period of time, and the second data includes a change in capacitance value associated with the user touching the insert that is above a capacitance threshold value.

In an embodiment, the task performed can be to prevent a false button-push event. For example, a false button-push event can be prevented by overriding a button-push event. The false button-push event can be prevented when the first data includes an actuation force that is above a force threshold value, and the second data includes a change in capacitance value associated with the user touching the insert that is below a capacitance threshold value.

In an embodiment, the second sensor can be a dry electrode, and the task performed can be to adjust an analog front-end (AFE) setting based on the first data and the second data. For example, the AFE setting can be adjusted when the second data indicates the presence of a user touch, or the second data indicates a touch event.

In an embodiment, the button assembly can further include a pressure-sensitive adhesive interposed between the insert and an exterior surface of the housing.

In an embodiment, the one or more processors can be part of a printed circuit board (PCB), and the computing device can further include a PCB adhesive interposed between the PCB and an interior side of the cavity.

In an embodiment, the second sensor can be an analog component that emits an analog electrical signal associated with a change in capacitance in response to the user touching the insert, and the second data obtained via the second sensor can be the analog electrical signal.

In an embodiment, the second sensor can be a digital component having a capacitive touch controller that transforms an analog signal into digital data. The analog signal can be associated with a change in capacitance in response to the user touching the insert. Additionally, the second data obtained via the second sensor can be digital data.

In an embodiment, the second data can include a touch location of the insert that is determined based on a change in capacitance and a time duration of the user touching the insert. Additionally, the task performed by the one or more processors can be further based on the touch location and the time duration.

In an embodiment, the housing can further include an upper side and a lower side. The lower side of the housing can be opposite to the upper side of the housing and can be configured to be in contact with a body part of the user when the user wears the wearable computing device. Additionally, a user interface can be on the upper side of the housing. Moreover, an optical sensor, coupled on the lower side of the housing, can be configured to detect reflected light from the body part of the user.

In an embodiment, the insert (e.g., button) can be a stainless-steel pill-shape button.

In an example embodiment, a computer-implemented method is provided. The computer-implemented method includes detecting, using a first sensor, actuation of an insert via an input provided by a user. The insert is at least partially positioned within a recess defined by a housing of the computing device. The method further includes detecting, using a second sensor, whether the user is touching the insert. Additionally, the method includes obtaining, from the first sensor, first data indicative of actuation of the insert. Moreover, the method includes obtaining, from the second sensor, second data indicative of the user touching the insert. Furthermore, the method includes performing, by one or more processors of the computing device, a task based, at least in part, on the first data and the second data.

In an embodiment, the method can further include determining that the first data includes an actuation force that is above a force threshold value for a specific period of time. Additionally, the method can include determining the second data includes a change in capacitance value associated with the user touching the insert that is above a capacitance threshold value. Moreover, the method can include resetting the computing device based on the determination that the actuation force is above the force threshold value for the specific period of time and the determination that the change in capacitance associated with the user touching the inset that is above the capacitance threshold value.

In an example embodiment, a non-transitory computer-readable medium which stores instructions that are executable by one or more processors of a computing device is provided. The non-transitory computer-readable medium stores instructions including instructions to cause the one or more processors to detect, using a first sensor, actuation of an insert via an input provided by a user, wherein the insert is at least partially positioned within a recess defined by a housing of computing device. The non-transitory computer-readable medium stores instructions to detect, using a second sensor, whether the user is touching the insert. The non-transitory computer-readable medium stores instructions to obtain, from the first sensor, first data indicative of actuation of the insert. Additionally, the non-transitory computer-readable medium can store instructions to obtain, from the second sensor, second data indicative of the user touching the insert. Furthermore, the non-transitory computer-readable medium stores instructions to cause the one or more processors to perform a task based, at least in part, on the first data and the second data.

These and other features, aspects, and advantages of various embodiments of the disclosure will become better understood with reference to the following description, drawings, and appended claims. The accompanying drawings, which are incorporated in and constitute a part of this specification, illustrate example embodiments of the disclosure and, together with the description, serve to explain the related principles.

### BRIEF DESCRIPTION OF THE DRAWINGS

Detailed discussion of example embodiments directed to one of ordinary skill in the art is set forth in the specification, which makes reference to the appended drawings, in which:

FIG. 1 depicts an example computing device according to according to one or more example embodiments of the disclosure;

FIG. 2 depicts a cutaway cross-section of the computing device of an example computing device according to one or more example embodiments of the disclosure:

FIG. 3 depicts an example block diagram of the computing device according to one or more example embodiments of the disclosure:

FIG. 4 depicts a top view of an example wearable computing device according to one or more example embodiments of the disclosure:

FIGS. 5 and 6 each depict a bottom view of a wearable computing device according to one or more embodiments of the disclosure:

FIG. 7 depicts a cross-sectional view of an example wearable computing device with an electrode having an electrical connection using a pin, according to one or more example embodiments of the disclosure:

FIG. 8 depicts a cross-sectional view of an example wearable computing device according to one or more example embodiments of the disclosure:

FIG. 9 depicts a cross-sectional view of an example wearable computing device with a button assembly according to one or more example embodiments of the disclosure:

FIG. 10 depicts different types of sensors in an example wearable computing device according to the disclosure; and

FIG. 11 depicts a flow diagram for performing a task using a multipurpose button according to one or more embodiments of the disclosure.

### DETAILED DESCRIPTION

Reference now will be made to embodiments of the disclosure, one or more examples of which are illustrated in the drawings. Each example is provided by way of explanation of the disclosure and is not intended to limit the disclosure. In fact, it will be apparent to those skilled in the art that various modifications and variations can be made to disclosure without departing from the scope or spirit of the disclosure. For instance, features illustrated or described as part of one embodiment can be used with another embodiment to yield a still further embodiment. Thus, it is intended that the disclosure covers such modifications and variations as come within the scope of the appended claims and their equivalents.

In some implementations, the computing device can include a button assembly having a plurality mode of sensing. In some instances, the button assembly can include an electrode that can function as a button. The electrode may form an insert positioned within a recess defined by a housing of the wearable computing device. For example, the button assembly can include a designated surface for button clicks as well as an electrode which could be used for touch sensing. Techniques described herein can improve force and signal transfer performance of the button assembly. Additionally, the techniques described herein can improve resistance to false and unintended triggering of the sensors by force. Moreover, the multi-purpose button assembly can improve resistance to false triggers from thermal shock stimuli. Furthermore, the multi-purpose button assembly can improve operational efficiency by optimizing power usage dynamically.

Examples embodiments in the disclosure provide several technical effects, benefits, and/or improvements in computing technology and the technology of computing devices. For instance, having one or more biometric sensor electrodes as part of the button assembly eliminates the need for a separate electrode to detect biometrics. For example, having one or more biometric sensor electrodes as part of the button assembly can allow for on-demand measurement of biometrics (e.g., electrocardiogram, electrodermal activity, etc.) of the user wearing the wearable computing device. Furthermore, the computing device may utilize less power than a conventional device by enabling the computing device to boot up from sleep mode based on data associated with the force applied to an insert of the button and the electrode determining a change in capacitance associated with a touch event. As yet another example, the computing device described herein can improve force and signal transfer performance of the button assembly by validating true push-button events with touch events. The different types of button assembly (e.g., using strain-based or ultrasonic sensors) can further improve force and signal transfer performance of the button assembly. Additionally, the techniques described herein can improve resistance to false and unintended triggering of the sensors by force. Moreover, the multi-purpose button can improve resistance to false triggers from thermal shock stimuli. Furthermore, the multi-purpose button can improve operational efficiency by optimizing power usage dynamically.

According to one or more examples of the computing device, the button assembly can allow for improvements in manufacturability, assembly and sustainability of computing devices. Previously designs integrated sensors into a larger housing with rigid adhesive. With the button assembly described herein, each component is separate, which enables manufacturing a computing device by using different material for performance or color, materials, finish design. Additionally, techniques described herein enable using sensors and flex assembly that are built independent to the housing, enable flexibility with adhesive or pressure-sensitive adhesive selection, enable improvements in manufacturing process and reparability, and enable improvements in device sustainability and recyclability.

Wearable computing devices having a button assembly according to example aspects of the present disclosure can provide numerous technical effects and benefits. According to one or more examples of the disclosure, the available space on a wearable computing device can be optimized since the electrode is part of the button assembly. Therefore, the wearable computing device according to the present disclosure may be smaller compared to conventional computing devices. Alternatively, or additionally, the wearable computing device may provide space for other components.

Conventional computing devices can use capacitive sensors (e.g., electrodes) to determine whether a user has touched the device by providing electrical signals for sensing. However, these capacitive sensors are typically positioned at locations (e.g., top, bottom) on a housing of the wearable computing device that are not suitable for having a button. Combining these two features into a single button assembly, using the techniques described herein, enables to solve problems related to button sensing and electrode sensing. The combination of the electrodes and the mechanical button allows the optimization of the limited surface area on the computing device by enabling the button to have added functionality, such as sensing for biometrics (e.g., electrocardiogram (ECG), electrodermal activity (EDA)) via the multipurpose sensor and button.

Examples of the disclosure are directed to a computing device. For example, the computing device can be a wearable computing device that can be worn, for example, on a user's wrist. The computing device can include a housing that has an exterior surface and an interior cavity. Additionally, the computing device can include a button assembly. The button assembly can include an insert forming a button that is positioned within a recess defined by the exterior of the housing. The button assembly can further include a first and second sensor. The button assembly can further include an electrode and a switch, the switch and the electrode being the first and the second sensor. The first sensor is used to detect actuation of the insert. The second sensor is used to detect a user touching the insert and/or for biometrics (e.g., ECG, EDA). The button assembly can be positioned at the periphery of the housing, such as the left or right side of the housing so that it is convenient for a user to press the button. A button adhesive can be interposed between the button and the exterior surface of the housing. The computing device can include different types of buttons, such as, but not limited to, a piezo button, a strain gauge button, or an ultrasonic sensing button. In some instances, the button can be a stainless-steel pill-shape button. For example, the button assembly can include a first sensor in the form of a mechanical sensor (e.g., switch) configured to detect actuation (e.g., pushing) of the insert via an input provided by a user. Additionally, the button assembly can further include a second sensor in the form of a biometric sensor (e.g., capacitive sensor, electrode, optical, ultrasonic) configured to detect whether the user is touching the insert.

The first sensor (e.g., force sensor), which is included in the button assembly, can be configured to detect a force applied to the insert. The second sensor (e.g., electrode), which can be part of the insert as a singular component, can be configured to measure a change in capacitance associated with a touch event. Additionally, the second sensor can be a dry electrode configured to measure one or more biometrics (e.g., ECG, EDA). In one embodiment, the second sensor can be an analog touch sensor. The analog touch sensor can respond to stimuli happening in its environment (e.g., touching of a user's finger) by emitting an electrical signal that corresponds to a change in capacitance following a touch event. Alternatively, in another embodiment, the second sensor can be a digital touch sensor. The digital touch sensor can translate the touch event into a digital signal. The digital touch sensor includes a capacitive touch controller that converts analog data from the electrode into digital data that can be processed by one or more processors of the computing device. The digital data can include information about the touch event (e.g., the touch location, time duration).

The computing device can include a printed circuit board (PCB) that is positioned inside the interior cavity of the housing. The PCB can include one or more processors configured to perform a task based on the force applied to the button and the change in capacitance associated with the electrode. For example, the PCB can reset the wearable computing device when: (i) the force applied to the button satisfied (e.g., is above) a force threshold value for at least a specific period of time; and (ii) the change in capacitance associated with the electrode satisfies a capacitance threshold value. Alternatively, the PCB can prevent a false button-push event (e.g., override a button-push event) when: (i) the force applied to the button satisfies (e.g., is above) a force threshold value for a specific period of time; and (ii) the change in capacitance associated with the electrode is below a capacitance threshold value. In yet another embodiment, when the electrode is a dry electrode, the PCB can adjust an analog front-end (AFE) setting when: (i) the force applied to the button satisfies (e.g., is above) a force threshold value; and (ii) the change in capacitance associated with the electrode satisfies (e.g., is above) a capacitance threshold value.

In some embodiments, the computing device can include a display screen on an upper side of the housing and an optical sensor on a lower side of the housing. The lower side of the housing is opposite to the upper side of the housing and is configured to be in contact with a body part of a user when the user wears the wearable computing device. The display screen includes a user interface. The optical sensor can be configured to detect a reflected light from the body part of the user. The optical sensor can obtain data indicative of the user's heart rate, heart rate variability, pulse rate, respiration rate, and/or blood oxygenation level. Additionally, some optical sensors utilize a camera and camera flash technology to illuminate an area on a user using the light output by the camera flash and capture the user's physiological data using the camera and/or an optical sensor coupled to the camera. For example, the optical sensor can be a photoplethysmography (PPG) sensor which may be used to monitor a heart rate of the user. The PPG sensor may include one or more emitters (e.g., light-emitting diodes (LEDs)) and a plurality of detectors (e.g., photodiodes). Light emitted from the one or more emitters is transmitted in a direction toward the user's body part (e.g., a portion of a user's wrist) which is in contact with the lower side of the housing. The light then interacts with blood vessels of the user, where it is modified to a degree that is influenced by the current blood volume in the blood vessels. The modified light is directed back toward the PPG detectors by reflection and/or refraction. The PPG detectors generate data (e.g., one or more signals) which is reflective of the current blood volume of the blood vessels of the user which received the light emitted from the one or more emitters.

As previously mentioned, the computing device can include a button adhesive interposed between the insert and the exterior surface of the housing. Moreover, the wearable computing device can further include a PCB adhesive interposed between the PCB and the interior cavity of the housing.

The computing devices can capture the user's biometric data (e.g., physiological data) by using capacitive and/or optical sensors. For example, the data outputted by the sensors can include touch data, motion data, and/or biometrics data. The touch data can include a touch event when a user has touched the sensor or is near the sensor. Additionally, some wearable computing devices may include a touch-sensitive display screen, while other wearable computing devices may include a non-touch-sensitive display screen. The motion data can include regarding the user's movements. The biometrics data obtained by measuring various biometric characteristics of the user, such as heart rate, perspiration levels, and the like.

Example of biometric data that can be captured by different sensors include data indicative of the user's heart rate, heart rate variability, pulse rate, respiration rate, and/or blood oxygenation level, electrocardiogram (ECG), electrodermal activity (EDA), and so on. For example, the ECG data include heart electrical activity data, and data that can be used to determine a likelihood of atrial fibrillation. Additionally, the EDA data can include the perspiration measurement data of a user's hands, which can be a marker for stress. Another example of a biometric sensor is a photoplethysmography (PPG) sensor. A problem with currently available fitness trackers worn on the user's wrist or chest is that they do not allow for the capture of biometric data from a finger and/or a fingertip of the user, where a relatively strong photoplethysmography (PPG) signal can be detected. Using the techniques described herein, the computing device can include a biometric sensor as part of the button assembly, which enables biometric data to be obtained from a user's finger or fingertip. Obtaining biometric data from a user's finger by using a wearable and portable device adds convenience and ease of access for a user.

In some implementations, the biometric sensor can include a coating on the insert. The thickness of a conductive material of the biometric sensor can be limited due, at least in part, to the coating. Furthermore, in such implementations, the sheet resistance of the biometric sensor can be limited due, at least in part, to the thickness of the conductive material of the biometric sensor being limited by the coating process.

In some implementations the wearable computing device can include a housing with an insert on an exterior side of the housing and a printed circuit board on an interior side of the housing. The button can include a force sensor and a biometric (e.g., touch) sensor. The force sensor can be configured to detect a force applied to the button. The touch sensor can be configured to measure a change in capacitance associated with the button. The printed circuit board can include one or more processors configured to perform a task based on the force applied to the button or the change in capacitance associated with the button.

The biometric sensor (e.g., touch sensor) can be a piezoelectric sensor, a strain gauge sensor, an ultrasonic sensor, or another similar biometric sensor. For example, the biometric sensor can be a piezoelectric sensor or piezo switch that is associated with the piezoelectric effect. The biometric sensor can include a piezoelectric element and an integrated semiconductor device. The charge generated by the piezoelectric element in the sensor can turn on the integrated semiconductor device causing the output of the sensor to be ‘on’ (e.g., active). When the integrated semiconductor device is on, current can flow through the integrated semiconductor device as with a conventional metal contact-based switch. After the voltage pulse is dissipated in the gate resistor, the integrated semiconductor device turns back off to its normal high impedance state. Additionally, the biometric sensor can further include a capacitor. The capacitor can be used to store the charge to lengthen the time constant of a gate circuit and lengthen the width of the pulse.

In the case of a piezo switch, the force could be compressive pressure that causes the piezo element to bend slightly. As a result, the piezo switches can produce a single, brief ‘on’ pulse. This pulse can vary with the amount of pressure applied or location of pressure applied on the button. For example, higher pressures generate higher voltages which take longer to dissipate. The touch data outputted by the button assembly can include both the amount of pressure applied and the location of the pressure applied to the button (e.g., insert). A piezo switch can have advantages over its conventional mechanical counterpart. One advantage is that there are no moving parts, just the small deformation on the front plate and the piezo element (e.g., typically a few micrometers). This enables that the lifetime of such a switch can be ensured to be tens of millions of operations, because there is no mechanical wear involved. Another advantage is that they may easily be completely sealed from the environment and thus made weatherproof.

According to some embodiments, the biometric sensor can be a strain gauge button. For example, the strain gauge can be a long, thin conductive strip in a zig-zag pattern of parallel lines. The strain gauge button is configured to measure strain on the button. A button can be an insert that is at least partially positioned within a recess defined by a housing of the wearable computing device. The strain gauge button can include an insulating flexible backing which supports a metallic foil pattern. The gauge is attached to the object by a suitable adhesive. As the object is deformed, the foil is deformed, causing its electrical resistance to change. The resistance change is related to the strain by the quantity known as the gauge factor. The strain gauge can take advantage of the physical property of electrical conductance and its dependence on the conductor's geometry. When an electrical conductor of the strain gauge button is stretched within the limits of its elasticity (e.g., such that it does not break or permanently deform), the conductor can become narrower and longer, which increases its electrical resistance end-to-end. Conversely, when a conductor is compressed, it will broaden and shorten, which decreases its electrical resistance end-to-end. From the measured electrical resistance of the strain gauge, the amount of induced stress can be inferred. The strain gauge button can be dependent on variations in temperature. Temperature change can cause a multitude of effects on the button. The button can change in size by thermal expansion, which will be detected as a strain by the gauge. Resistance of the gauge will change, and resistance of the connecting wires will change.

An ultrasonic sensor is an instrument that measures the distance to an object using ultrasonic sound waves. An ultrasonic sensor uses a transducer to send and receive ultrasonic pulses that relay back information about an object's proximity. For example, ultrasonic imaging of the forearm could be used to recognize gestures (e.g., hand gestures, finger gestures).

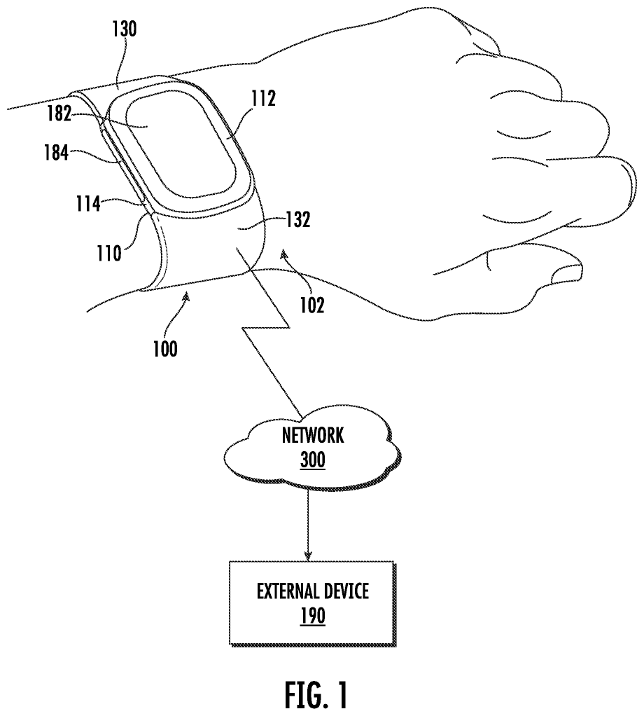

Referring now to the drawings, FIGS. 1 through 4 illustrate examples of a computing device 100 (e.g., wearable computing device, mobile device, laptop, tablet). according to examples of the disclosure. FIG. 1 illustrates an example computing device 100 which can be a wearable computing device. The wearable computing device can be worn, for example, on an arm 102 (e.g., wrist) of a user. The computing device 100 includes a housing 110 defining a cavity 112. Additionally, the computing device 100 includes a button assembly having an insert 184 (e.g., a button) that is partially positioned within a recess defined by an exterior surface of the housing 110. The insert 184 is positioned at a periphery 114 (e.g., left-side, right-side, edge) of the housing 110. Moreover, the button assembly includes a plurality of sensors (e.g., strain sensor, ultrasonic sensor, and a force sensor) configured to detect actuation of the button via an input provided by a user and whether the user is touching the button. The computing device 100 includes one or more processors 150 positioned within the cavity 112.

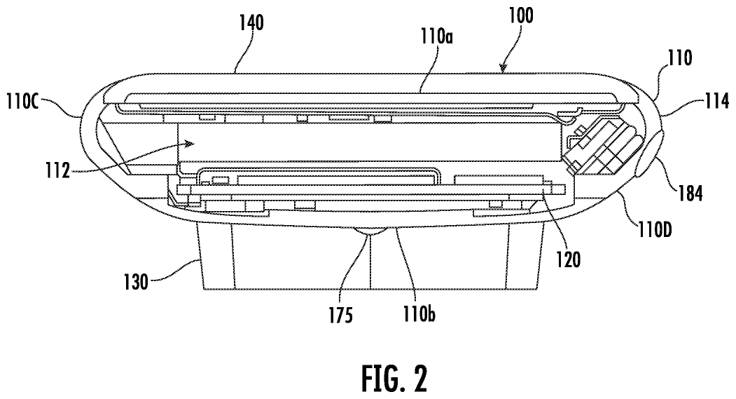

FIG. 2 illustrates a cutaway cross-section view of the computing device 100 where the housing 110 includes an upper side 110a and a lower side 110b, left side 110c, right side 110d. The insert 184 can be positioned on the left side 110c or right side 110d of the housing 110. Additionally, the insert 184 can be positioned at the periphery 114 of the housing.

In an embodiment, the computing device 100 can be a wearable computing device, and the insert 184 can be positioned at a periphery 114 of the housing. In another embodiment, the computing device can be a mobile device, and the insert 184 can be positioned at a periphery 114 of the housing of the mobile device or on the upper side 110a of the housing of the mobile device. In another embodiment, the computing device can be a laptop, and the insert 184 can be positioned at a periphery 114 of the housing of the laptop or on a key board of the laptop. In yet another embodiment, the computing device can be a computer tablet, and the insert 184 can be positioned at a periphery 114 of the housing of the computer tablet or on the upper side 110a of the housing of the computer tablet.

The housing 110 defines a cavity 112 in which one or more electronic components (e.g., disposed on one or more printed circuit boards) are disposed. For example, the computing device 100 can include a printed circuit board 120 disposed within the cavity 112. Furthermore, one or more electronic components can be disposed on the printed circuit board 120. The computing device 100 can further include a battery (not shown) that is disposed within the cavity 112 defined by the housing 110. The computing device 100 further includes various sensors 170 that are part of the button assembly that also includes the insert 184. For example, the sensors 170 can be biometric sensors such as piezoelectric sensors, strain gauge sensors, ultrasonic sensors, and other similar biometric sensors. In some instances, the sensors 170 may include a first sensor 172 (e.g., force sensor) and a second sensor 174 (e.g., touch sensor). The second sensor 174 can be positioned on the surface of the insert 184.

The computing device 100 can also include optical sensors. For example, the second sensor 174 can include one or more optical sensors. The optical sensors can be photoplethysmography (PPG) sensors which may be used to monitor a heart rate of the user. The optical sensors may include one or more emitters (e.g., light-emitting diodes (LEDs)) and a plurality of detectors (e.g., photodiodes). Light emitted from the one or more emitters is transmitted in a direction toward the user's body part (e.g., a portion of a user's wrist) which is in contact with the lower side 110b of the housing 110. The light then interacts with blood vessels of the user, where it is modified to a degree that is influenced by the current blood volume in the blood vessels. The modified light is directed back toward the PPG detectors by reflection and/or refraction. The PPG detectors generate data (e.g., one or more signals) which is reflective of the current blood volume of the blood vessels of the user which received the light emitted from the one or more emitters.

Additionally, the computing device 100 can include motion sensors (not shown) positioned within the cavity 112 of the housing 110. For example, the motion sensors may also include an accelerometer 174 which may be used to capture motion data indicative of motion of the computing device 100. Alternatively, or additionally, the motion sensors may also include a gyroscope 176 which may also be used to capture motion information with respect to the computing device 100.

The computing device 100 can include a first band 130 and a second band 132. As shown, the first band 130 can be coupled to the housing 110 at a first location thereon. Conversely, the second band 132 can be coupled to the housing 110 at a second location thereon. Furthermore, the first band 130 and the second band 132 can be coupled to one another to secure the housing 110 to the arm 102 of the user.

In some examples, the first band 130 can include a buckle or clasp (not shown). Additionally, the second band 132 can include a plurality of apertures (not shown) spaced apart from one another along a length of the second band 132. In such implementations, a prong of the buckle associated with the first band 130 can extend through one of the plurality of openings defined by the second band 132 to couple the first band 130 to the second band 132. It should be appreciated that the first band 130 can be coupled to the second band 132 using any suitable type of fastener. For example, in some implementations, the first band 130 and the second band 132 can include a magnet. In such implementations, the first band 130 and the second band 132 can be magnetically coupled to one another to secure the housing 110 to the arm 102 of the user.

The computing device 100 can include a cover 140 positioned on the housing 110 so that the cover 140 is positioned on top of (over) the display screen 182. In this manner, the cover 140 can protect the display screen 182 from being scratched. In some implementations, the computing device 100 can include a seal (not shown) positioned between the housing 110 and the cover 140. For instance, a first surface of the seal can contact the housing 110 and a second surface of the seal can contact the cover 140. In this manner, the seal between the housing 110 and the cover 140 can prevent a liquid (e.g., water) from entering the cavity 112 defined by the housing 110.

It should be understood that the cover 140 can be optically transparent so that the user can view information being displayed on the display screen 182. For instance, in some implementations, the cover 140 can include a glass material. It should be understood, however, that the cover 140 can include any suitable optically transparent material.

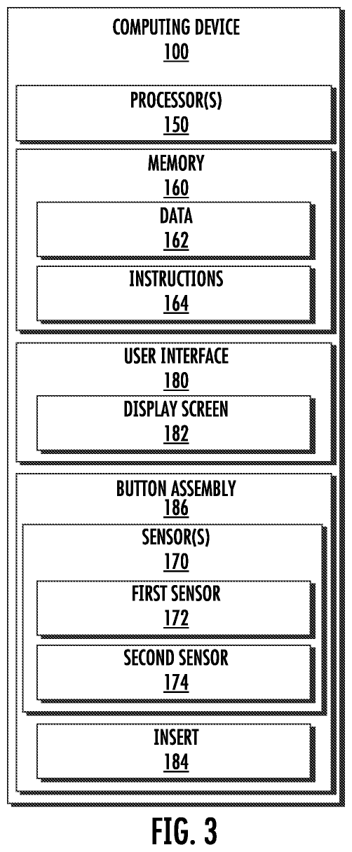

FIG. 3 illustrates an example block diagram of the computing device 100 according to one or more example embodiments of the disclosure. The computing device 100 may include one or more processors 150, one or more memory devices 160, a user interface 180, and one or more button assembly 186.

For example, the one or more processors 150 can be any suitable processing device that can be included in a computing device 100. For example, such a processor 150 may include one or more of a processor, processor cores, a controller and an arithmetic logic unit, a central processing unit (CPU), a graphics processing unit (GPU), a digital signal processor (DSP), an image processor, a microcomputer, a field programmable array, a programmable logic unit, an application-specific integrated circuit (ASIC), a microprocessor, a microcontroller, etc., and combinations thereof, including any other device capable of responding to and executing instructions in a defined manner. The one or more processors 150 can be a single processor or a plurality of processors that are operatively connected, for example in parallel.

The memory 160 can include one or more non-transitory computer-readable storage mediums, such as such as a Read Only Memory (ROM), Programmable Read Only Memory (PROM), Erasable Programmable Read Only Memory (EPROM), and flash memory, a USB drive, a volatile memory device such as a Random Access Memory (RAM), a hard disk, floppy disks, a blue-ray disk, or optical media such as CD ROM discs and DVDs, and combinations thereof. However, examples of the memory 160 are not limited to the above description, and the memory 160 may be realized by other various devices and structures as would be understood by those skilled in the art.

For example, memory 160 can store instructions, that when executed, cause the one or more processors 150 to obtain, via the first sensor 172 (e.g., force sensor), first data indicative of actuation of the insert 184, and obtain, via the second sensor 174 (e.g., touch sensor) second data indicative of the user touching the insert. Additionally, the one or more processors 150 can perform a task based, at least in part, on the first data and the second data, as described according to examples of the disclosure. The first data and the second data can be stored as data 162 in memory 160. For example, memory 160 can store instructions, that when executed, cause the one or more processors 150 to execute one or more functions of the computing device 100 based on the first data and the second data obtained from the sensors 170, as described according to examples of the disclosure.

Memory 160 can also include data 162 and instructions 164 that can be retrieved, manipulated, created, or stored by the one or more processor(s) 150. In some example embodiments, such data can be accessed and used as input to perform a task. The data 162 can include force data, optical data, touch data, and other data obtained from the first sensor 172 and/or the second sensor 174. In some examples, the memory 160 can include data used to perform one or more processes and instructions that execute one or more functions of the computing device 100 based on the data 162.

The computing device 100 can include a user interface 180 configured to receive an input from a user by the user applying a force to the user interface (e.g., via a button assembly 186, a thumb, finger, or an input device such as a stylus or pen). The computing device 100 may execute a function in response to receiving the input from the user (e.g., checking health information about the user such as a blood pressure, making and/or receiving a phone call, sending and/or receiving a text message, obtaining a current time, setting a timer, a stopwatch function, controlling an external device such as a home appliance, and the like).

For example, the computing device 100 may be connected to one or more external devices 190 in a wireless and/or wired manner. The computing device 100 may be connected to the external device 190 over a network 300 such as a local area network (LAN), wireless local area network (WLAN), wide area network (WAN), personal area network (PAN), virtual private network (VPN), or the like. For example, wireless communication between the computing device 100 and the external device 190 may be performed via a wireless LAN, Wi-Fi, Bluetooth, ZigBee, Wi-Fi direct (WFD), ultra-wideband (UWB), infrared data association (IrDA), Bluetooth low energy (BLE), near field communication (NFC), a radio frequency (RF) signal, and the like. For example, the wired communication connection may be performed via a USB cable, a pair cable, a coaxial cable, an optical fiber cable, an Ethernet cable, and the like.

The user interface 180 may include a display screen 182 which displays information viewable by the user (e.g., time, date, biometric information, notifications, etc.). For example, the display screen 182 may be a non-touch sensitive display screen. The display screen 182 may include a liquid crystal display (LCD), a light emitting diode (LED) display, an organic light emitting diode (OLED) display, active-matrix organic light emitting diode (AMOLED), flexible display, 3D display, a plasma display panel (PDP), a cathode ray tube (CRT) display, and the like, for example. However, the disclosure is not limited to these example displays and may include other types of displays. That is, the non-touch sensitive display screen 182 may not be capable of sensing a touch event via conduction using electrical conductors (e.g., like a capacitive touch screen or resistive screen) to change a capacitance or resistance of a circuit. Thus, the non-touch sensitive display screen 182 does not include a touch panel which may include conductive layers and/or resistive layers of circuitry. In some implementations, the display screen 182 may have a square or rectangular shape, or may be annular in shape (e.g., elliptical, circular, etc.). However, it should be appreciated that the display screen 182 can have any suitable shape.

The computing device 100 can include one or more button assembly 186. In some instances, the button assembly can include a first sensor 172, a second sensor 174, and an insert 184. The insert 184 (.g., button) can be at least partially positioned within a recess defined by a housing of the computing device. The insert 184 can have biometric sensors, such as a piezoelectric sensor, a strain gauge sensor, and an ultrasonic sensor.

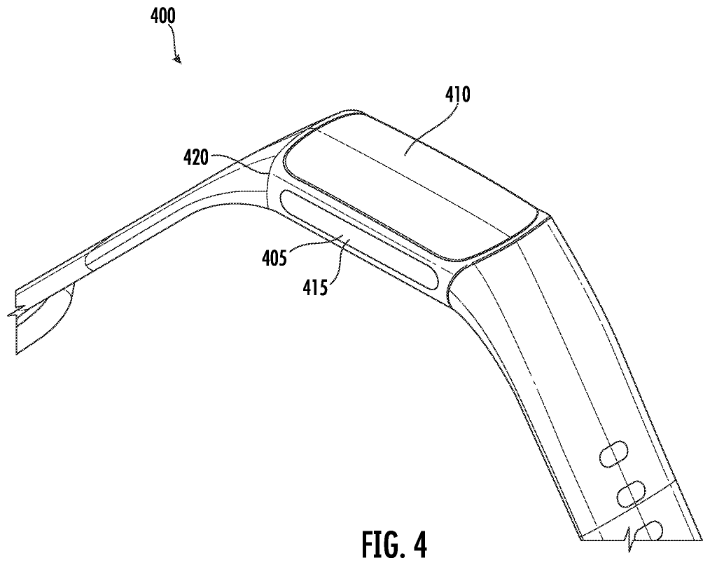

FIG. 4 depicts a top view of an example wearable computing device 400 according to example embodiments of the disclosure. The wearable computing device 400 includes a housing 420, an electrode 415 (a “second sensor”) positioned on the sidewall of the housing, and a display 410 positioned on the top of the housing 420. The electrode 415 can also function as an insert 405 (e.g., a button) that is connected with a force sensor (a “first sensor”), which enables multifunctionality as discussed in some of the embodiments described herein.

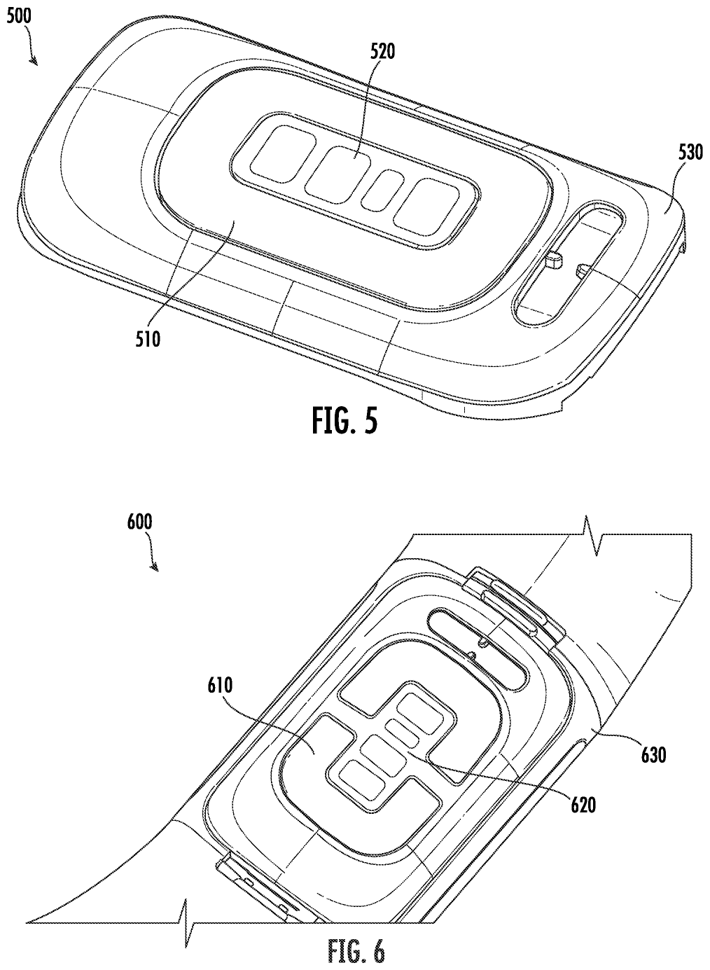

FIGS. 5 and 6 each depict a bottom view of a wearable computing device according to one or more embodiments of the disclosure. The wearable computing device 500, 600 can include one or more electrodes 510, 610 and sensors (e.g., optical sensors 520, 620). In some instances, the housing 530, 630 can be a translucent plastic molding. The translucent plastic molding can be thin plastic sheet that is optically clear to protect the optical sensors 520, 620. The lower side of the housing is configured to be in contact with a body part of a user when the user wears the wearable computing device. The translucent mold enables light emitted from the optical sensor 520, 620 to reflect off the user's skin. As previously discussed, the optical sensors 520, 620 can be configured to detect reflected light from the body part of the user. The optical sensor can be captured by different optical sensors including data indicative of the user's heart rate, heart rate variability, pulse rate, respiration rate, and/or blood oxygenation level. Additionally, some optical sensors utilize a camera and camera flash technology to illuminate an area on a user using the light output by the camera flash and capture the user's physiological data using the camera and/or an optical sensor coupled to the camera. For example, the optical sensor can be a PPG sensor which may be used to monitor a heart rate of the user.

The electrodes 510, 610 positioned at the lower side of the housing 530, 630 can be a metal material. Additionally, the electrodes 510, 610 can be a plurality of shapes. For example, as illustrated in FIG. 5, the electrodes 510 can be one continuous metal part that has a racetrack-like shape around the optical sensors 520. In another example, as illustrated in FIG. 6, the electrodes 610 can be two metal parts that are U-shape. The different shapes of the electrodes 510, 610 allow for different functionality and features. For example, when the electrodes 510 are one singular part, then the electrodes 510 can perform spot-check EDA measurements based on a user's request or input. The EDA value can be insights of a user's stress-level. Alternatively, when the electrodes 610 are two metal parts, then the electrodes 610 can perform continuous EDA measurements, without the need of a user's request or input. Additionally, when the electrodes 610 are two metal parts, the electrodes 610 can also do spot-check EDA measurements based on a user's request.

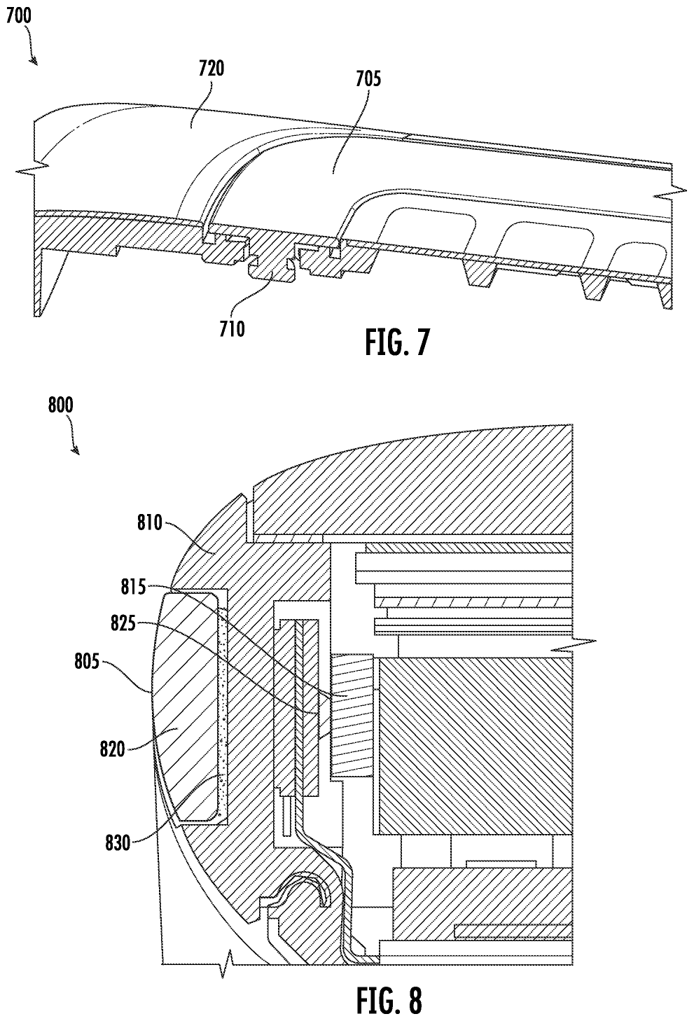

FIG. 7 depicts a cross-sectional view of an example wearable computing device 700 with an electrode 705 having an electrical connection using a pin, according to one or more example embodiments of the disclosure. The electrode 705 can be positioned at the lower side of the housing 720, as previously illustrated in FIGS. 5 and 6. The electrode 705, which can be a metal part, can also include a metal pin 710 to connect to the internal hardware (not shown) of the wearable computing device 700. The metal pin 710 allows for an electrical connection between the electrode 705 and the hardware of the wearable computing device 700. The hardware can be positioned inside the housing 720. The hardware can include, but is not limited to, a printed circuit board having one or more processors.

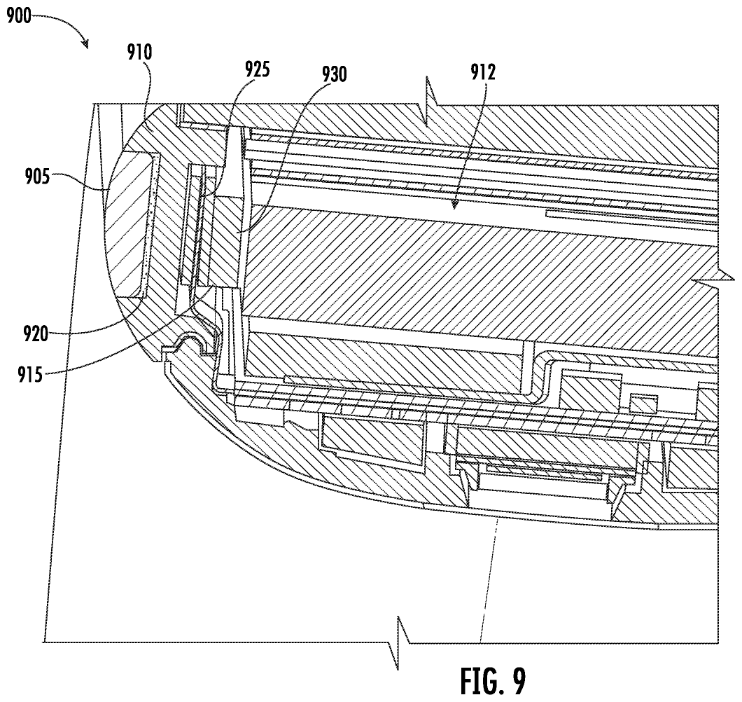

FIG. 8 depicts a cross-sectional view of an example wearable computing device 800 according to example embodiments of the disclosure. The electrode 820 (the “second sensor”) is positioned on the side wall of the housing 810 of the computing wearable device 800. The electrode 820 also functions as a button, as described using techniques described herein. In conventional systems, the electrode 820 may have been positioned on the upper side of the housing, but this position is not functional for the electrode 820 to also function as a button. By having the electrode 820 positioned on the side wall of the housing, as depicted in FIG. 8, it can be intuitive for a user to also use the electrode 820 as a button. Additionally, given that the button 805 is also an electrode 820, the wearable computing device 800 can have built-in intelligence and feedback to the user. For example, when a biometric (e.g., EDA, ECG) measurement is being obtained by the electrode 820, the electrode 820 can indicate to the PCB of the wearable computing device 800 that the user is not pressing the electrode 820 correctly (e.g., not pressing hard enough, pressing too hard, pressing at the wrong location) in order to obtain a good biometric reading. In some instances, based on an output of the electrode 820, the wearable computing device 800 can determine that the user is not pressing the electrode 820 correctly, and present on the user interface 180 (FIG. 1) a notification that the user is not applying enough force when pressing the electrode 820 or not pressing the electrode 820 at the proper location.

In some implementations, the electrode 820 can be a strain sensor that is made on a chip-scale package (CSP) that is mounted on the PCB. By using a strain gauge sensor, the wearable computing device 800 can have multiple layers of materials (e.g., electrode 820, button 805, button adhesive 830, housing 810, pin 825, force sensor 815). As a result, the electric components (e.g., force sensor 815, pin 825) can be positioned inside the housing 810, which enables the wearable computing device 800 to have improved durability and increased water resistance given that some of the electric components are sealed inside the housing 810. In this example, when the electrode 820, which also functions as a button 805, is coupled to the pin 825. The pin 825 can create an electrical connection to the PCB for the electrical functionality of the electrode 820. Additionally, when the electrode 820 and button 805 are pressed, the pin 825 exerts a force against the force sensor 815 (the “first sensor”). The force sensor 815 can be configured to output a force value to the PCB of the wearable computing device 800. In some instances, the force sensor 815 can be a force sensing layer, where the resistors are within the layers of the PCB of the computing wearable device 800.

FIG. 9 depicts a cross-sectional view of an example wearable computing device 900 with a button assembly according to example embodiments of the disclosure. The wearable computing device 900 includes a housing 910 defining a cavity 912. The wearable computing device 900 includes a button assembly having an insert 905 (e.g., button) that is at least partially positioned within a recess defined by the housing 910. The button assembly includes a first sensor 930) and a second sensor 940. The first sensor 930 can be a force sensor (e.g., force sensor 815 in FIG. 8) configured to detect actuation of the insert 905 via an input provided by a user. The second sensor 940 can be an electrode (e.g., electrode 820 in FIG. 8) configured to detect whether the user is touching the insert. The second sensor 940 can be electrically connected to a PCB 915 via a pin (e.g., pin 825 in FIG. 8). The PCB 915 (e.g., one or more processors) can be positioned within the cavity 912, and can be configured to obtain, via the first sensor 930, first data indicative of actuation of the insert. Additionally, the PCB 915 can be configured to obtain, via the second sensor 940, second data indicative of the user touching the insert. Furthermore, the PCB 915 can be configured to perform a task based, at least in part, on the first data and the second data. For example, the task can be measuring an electrocardiogram (ECG) reading and an electrodermal activity (EDA) reading.

In some implementations, the second sensor 940 (e.g., electrode) can be part of the insert 905 (e.g., button). For example, the second sensor 940 can also function as the insert 905, without requiring an additional mechanical device. The second sensor 940 can be a strain gauge sensor. In some instances, the second sensor 940 can be a strain gauge sensor and an ultrasonic sensor. The second sensor 940 can be a capacitive sensor (e.g., dry electrode) that is electrically connected to the PCB 915 via a pin. The second sensor 940 and the insert 905 can be positioned at a periphery (e.g., side wall) of the housing 910.

In some implementations, the button assembly further includes a pressure-sensitive adhesive 920 interposed between the insert 905 and an exterior surface of the housing 910. The pressure-sensitive adhesive 920 can be soft, compared to conventional systems (e.g., which used rigid superglue adhesive), which makes it easier to remove individual components to enable improvements in device recyclability. Additionally, the button assembly further includes a PCB adhesive 925 interposed between the PCB 915 and an interior side of the cavity 912.

In some implementations, the second sensor 940 can be an analog component that emits an analog electrical signal associated with a change in capacitance in response to the user touching the insert, and the second data obtained by the PCB 915 is the analog electrical signal. In other implementations, the second sensor can be a digital component having a capacitive touch controller that transforms an analog signal associated with a change in capacitance in response to the user touching the insert into digital data, and the second data obtained by the PCB 915 is the digital data. The digital data can include a touch location of the insert that is determined based on a change in capacitance and a time duration of the user touching the insert, and the task performed is further based on the touch location and the time duration.

In some implementations, the insert 905 is a stainless-steel pill-shape button. By having a pill shape insert 905, the quality of force transfer is symmetric which results in improvement in the force and signal transfer performance of the button. Based on the techniques described herein, when a force is applied to the second sensor 940 (e.g., electrode), the force can be transferred to the first sensor 930 (e.g., force sensor).

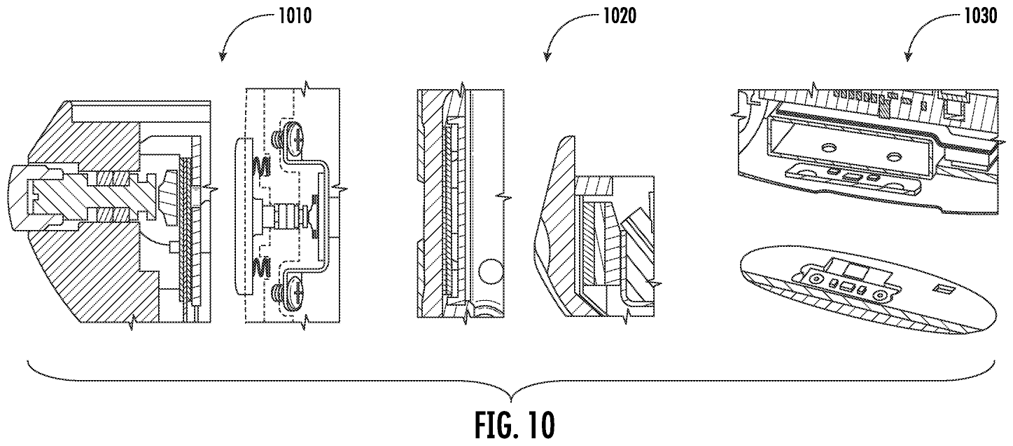

FIG. 10 depicts different types of sensors in an example wearable computing device according to the disclosure. Conventional systems may use a mechanical button 1010 or an inductive button 1020. When conventional systems use an inductive button 1020, the system has a gap between the button and the housing, which makes it susceptible to false actuation. Additionally, induction button 1020 can have a high number of components, performance can be dependent on part tolerance, and has a medium to high power consumption.

According to some embodiments, using the techniques described herein, components of the button assembly 186 of the computing device 100 can be a strain button 1030, an ultrasonic button, and/or an infrared button. The ultrasonic button can improve functionality by enabling a user to scroll using the ultrasonic button. The infrared button can be an infrared button that is configured to confirm that a user is pressing the button when the infrared light emitted from the infrared button is blocked.

In some implementations, by using the strain button 1030, the second sensor (e.g., second sensor 940 in FIG. 9, electrode) described herein consume less power (e.g., power consumption is about 10 μA @100 Hz scan rate) than the inductive button 1020 (e.g., power consumption is about 80 μA @40 Hz scan rate), and therefore the second sensor can be powered on even when the device is shut down. As a result, the computer wearable device can be powered-on, reset, and booted up by pressing the insert (e.g., insert 905 in FIG. 9), in a similar method as with the mechanical button 1010. Conventional systems may use this functionality with the mechanical button 1010 because the mechanical button 1010 does not consume power. However, mechanical buttons take additional space, have mechanical wear, have limited modes of interaction, and have a high number of components, among other issues.

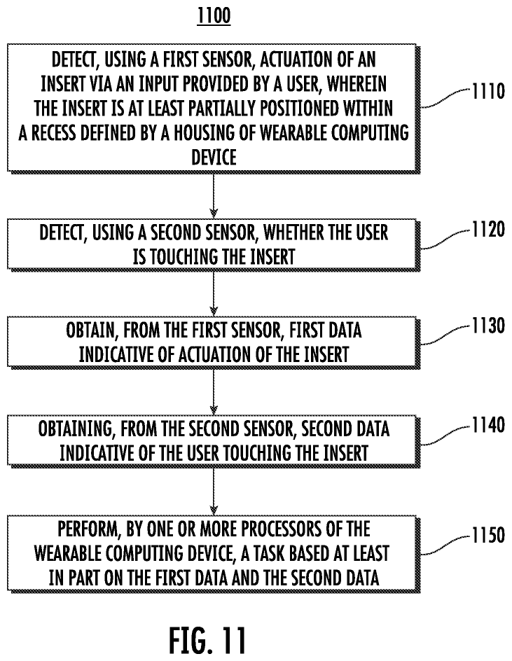

Examples of the disclosure are also directed to computer implemented methods of a wearable computing device. FIG. 11 each illustrate a flow diagram of an example, non-limiting computer-implemented method according to one or more example embodiments of the disclosure.

Referring to FIG. 11, in an example computer implemented method 1100 at operation 1110 one or more processors 150 can detect, using a first sensor (e.g., first sensor 930 in FIG. 9), actuation of an insert (e.g., insert 905 in FIG. 9) via an input provided by a user. The insert is at least partially positioned within a recess defined by a housing of a computing device. The one or more processors 150 can be part of a PCB (e.g., PCB 915 in FIG. 9). FIG. 11 describes example tasks that can be performed based on the first data and the second data.

At operation 1120, the one or more processors 150 detects, using a second sensor (e.g., second sensor 940 in FIG. 9), whether the user is touching the insert.

At operation 1130, the one or more processors 150 obtains, from the first sensor, first data indicative of actuation of the insert.

At operation 1140, the one or more processors 150 obtains, from the second sensor, second data indicative of the user touching the insert.

At operation 1150, the one or more processors 150 performs a task based at least in part on the first data and the second data. Example tasks can include measuring an electrocardiogram (ECG) reading and an electrodermal activity (EDA) reading. Additionally, the example tasks can include rebooting the computing device, resetting the computing device, powering on the computing device, powering down the computing device. Moreover, the example tasks can include checking health information about the user such as a blood pressure, making and/or receiving a phone call, sending and/or receiving a text message, obtaining a current time, setting a timer, a stopwatch function, controlling an external device 190 such as a home appliance, an electronic device such as a television, and the like.

Additionally, based on the techniques described herein that allows for the second sensor (e.g., electrode) to also function as the insert, the first data and/or the second data can include additional input information not normally available in conventional systems. In some instances, the first data and/or the second data can include multiple levels of force measurement exerted on the insert. For example, multiple levels of force measurement can be a first level of force measurement, a second level of force measurement, a third level of force measurement, and so on, where the first level is greater than the second level, and the second level is greater than the third level of force. Moreover, the first data and/or second data can include a double-click of the insert. Furthermore, the first data and/or the second data can include a time duration of the force exerted on the insert, and a button location associated with a location of the force exerted on the insert.

In some implementations, the method 1100 can further include determining that the first data includes an actuation force that is above a force threshold value for a specific period of time. Additionally, the method 1100 can include determining the second data includes a change in capacitance value associated with the user touching the insert that is above a capacitance threshold value. Moreover, the method 1100 can include resetting the computing device based on the determination that the actuation force is above the force threshold value for the specific period of time and the determination that the change in capacitance associated with the user touching the inset that is above the capacitance threshold value.

In some implementations, the method 1100 can further include determining that the first data includes an actuation force that is above a force threshold value. Additionally, the method 1100 can include determining that the second data includes a change in capacitance value associated with the user touching the insert that is below a capacitance threshold value. Moreover, the method 1100 can include overriding a push-button event when based on a determination that the push-button event is a false button-push event.

In some implementations, the method 1100 can further include adjusting an analog front-end (AFE) setting based on the first data and the second data. For example, the second sensor can be a dry electrode that can determine additional input information from a user associated with the insert.

Aspects of the above-described example embodiments may be recorded in non-transitory computer-readable media including program instructions to implement various operations embodied by a computer. The media may also include, alone or in combination with the program instructions, data files, data structures, and the like. Examples of non-transitory computer-readable media include magnetic media such as hard disks, floppy disks, and magnetic tape: optical media such as CD ROM disks, Blue-Ray disks, and DVDs: magneto-optical media such as optical discs; and other hardware devices that are specially configured to store and perform program instructions, such as semiconductor memory, read-only memory (ROM), random access memory (RAM), flash memory, USB memory, and the like. Examples of program instructions include both machine code, such as produced by a compiler, and files containing higher level code that may be executed by the computer using an interpreter. The program instructions may be executed by one or more processors. The described hardware devices may be configured to act as one or more software modules in order to perform the operations of the above-described embodiments, or vice versa. In addition, a non-transitory computer-readable storage medium may be distributed among computer systems connected through a network and computer-readable codes or program instructions may be stored and executed in a decentralized manner. In addition, the non-transitory computer-readable storage media may also be embodied in at least one application specific integrated circuit (ASIC) or Field Programmable Gate Array (FPGA).

Each block of the flowchart illustrations may represent a unit, module, segment, or portion of code, which comprises one or more executable instructions for implementing the specified logical function(s). It should also be noted that in some alternative implementations, the functions noted in the blocks may occur out of order. For example, two blocks shown in succession may in fact be executed substantially concurrently (simultaneously) or the blocks may sometimes be executed in the reverse order, depending upon the functionality involved.

While the disclosure has been described with respect to various example embodiments, each example is provided by way of explanation, not limitation of the disclosure. Those skilled in the art, upon attaining an understanding of the foregoing, can readily produce alterations to, variations of, and equivalents to such embodiments. Accordingly, the disclosure does not preclude inclusion of such modifications, variations and/or additions to the disclosed subject matter as would be readily apparent to one of ordinary skill in the art. For example, features illustrated or described as part of one embodiment can be used with another embodiment to yield a still further embodiment. Thus, it is intended that the disclosure covers such alterations, variations, and equivalents.

Terms used herein are used to describe the example embodiments and are not intended to limit and/or restrict the disclosure. The singular forms “a.” “an” and “the” are intended to include the plural forms as well, unless the context clearly indicates otherwise. In this disclosure, terms such as “including”. “having”. “comprising.” and the like are used to specify features, numbers, steps, operations, elements, components, or combinations thereof, but do not preclude the presence or addition of one or more of the features, elements, steps, operations, elements, components, or combinations thereof.

It will be understood that, although the terms first, second, third, etc., may be used herein to describe various elements, the elements are not limited by these terms. Instead, these terms are used to distinguish one element from another element. For example, without departing from the scope of the disclosure, a first element may be termed as a second element, and a second element may be termed as a first element.

The term “and/or” includes a combination of a plurality of related listed items or any item of the plurality of related listed items. For example, the scope of the expression or phrase “A and/or B” includes the item “A”, the item “B”, and the combination of items “A and B.”

In addition, the scope of the expression or phrase “at least one of A or B” is intended to include all of the following: (1) at least one of A, (2) at least one of B, and (3) at least one of A and at least one of B. Likewise, the scope of the expression or phrase “at least one of A, B, or C” is intended to include all of the following: (1) at least one of A, (2) at least one of B, (3) at least one of C, (4) at least one of A and at least one of B, (5) at least one of A and at least one of C, (6) at least one of B and at least one of C, and (7) at least one of A, at least one of B, and at least one of C.

1. A computing device comprising:
a housing defining a cavity;
a button assembly comprising:
an insert at least partially positioned within a recess defined by the housing;
a first sensor configured to detect actuation of the insert via an input provided by a user; and
a second sensor configured to detect whether the user is touching the insert;
one or more processors positioned within the cavity, the one or more processors configured to:
obtain, via the first sensor, first data indicative of actuation of the insert;
obtain, via the second sensor, second data indicative of the user touching the insert; and
perform a task based, at least in part, on the first data and the second data.

2. The computing device of claim 1, wherein the insert and the second sensor are one singular component.

3. The computing device of claim 1, wherein the first sensor comprises a force sensor, and wherein the second sensor comprises a strain sensor.

4. The computing device of claim 1, wherein the second sensor further comprises an ultrasonic sensor.

5. The computing device of claim 1, wherein the computing device is a wearable computing device, and wherein the insert is positioned at a periphery of the housing.

6. The computing device of claim 1, wherein the computer device is a mobile device or a laptop.

7. The computing device of claim 1, wherein the task is to measure an electrocardiogram (ECG) reading and an electrodermal activity (EDA) reading.

8. The computing device of claim 1, wherein the task is resetting the computing device when:
the first data includes an actuation force that is above a force threshold value for at least a specific period of time; and
the second data includes a change in capacitance value associated with the user touching the insert that is above a capacitance threshold value.

9. The computing device of claim 1, wherein the task is overriding a button-push event when:
the first data includes an actuation force that is above a force threshold value; and
the second data includes a change in capacitance value associated with the user touching the insert that is below a capacitance threshold value.

10. The computing device of claim 1, wherein the second sensor is a dry electrode, and wherein the task is adjusting an analog front-end (AFE) setting based on the first data and the second data.

11. The computing device of claim 1, wherein the button assembly further comprises a pressure-sensitive adhesive interposed between the insert and an exterior surface of the housing.

12. The computing device of claim 1, the one or more processors are part of a printed circuit board (PCB), and the computing device further comprising:
a PCB adhesive interposed between the PCB and an interior side of the cavity.

13. The computing device of claim 1, wherein the second sensor is an analog component that emits an analog electrical signal associated with a change in capacitance in response to the user touching the insert, and wherein the second data is the analog electrical signal.

14. The computing device of claim 1, wherein the second sensor is a digital component having a capacitive touch controller that transforms an analog signal associated with a change in capacitance in response to the user touching the insert into digital data, wherein the second data is the digital data.

15. The computing device of claim 1, wherein the second data includes a touch location of the insert that is determined based on a change in capacitance and a time duration of the user touching the insert, and wherein the task performed is further based on the touch location and the time duration.

16. The computing device of claim 1, wherein the housing further includes an upper side and a lower side, wherein the lower side of the housing is opposite to the upper side of the housing and is configured to be in contact with a body part of the user when the computing device is worn by the user;
a user interface on the upper side of the housing; and
an optical sensor, coupled on the lower side of the housing, configured to detect a reflected light from the body part of the user.

17. The computing device of claim 1, wherein the insert is a stainless-steel pill-shape button.

18. A computer-implemented method, comprising:
detecting, using a first sensor, actuation of an insert via an input provided by a user, wherein the insert is at least partially positioned within a recess defined by a housing of computing device;
detecting, using a second sensor, whether the user is touching the insert;
obtaining, from the first sensor, first data indicative of actuation of the insert;
obtaining, from the second sensor, second data indicative of the user touching the insert; and
performing, by one or more processors of the computing device, a task based, at least in part, on the first data and the second data.

19. The computer-implemented method of claim 18, further comprising:
determining that the first data includes an actuation force that is above a force threshold value for a specific period of time;
determining the second data includes a change in capacitance value associated with the user touching the insert that is above a capacitance threshold value; and
resetting the computing device based on the determination that the actuation force is above the force threshold value for the specific period of time and the determination that the change in capacitance associated with the user touching the inset that is above the capacitance threshold value.

20. A non-transitory computer-readable medium which stores instructions that are executable by one or more processors of a computing device, the instructions comprising:
detect, using a first sensor, actuation of an insert via an input provided by a user, wherein the insert is at least partially positioned within a recess defined by a housing of computing device;
detect, using a second sensor, whether the user is touching the insert;
obtain, from the first sensor, first data indicative of actuation of the insert;
obtain, from the second sensor, second data indicative of the user touching the insert; and
instructions to cause the one or more processors to perform a task based, at least in part, on the first data and the second data.
